(19) United States
(12) Patent Application Publication
(10) Pub. No.: US 2008/0027384 A1
(43) Pub. Date: Jan. 31, 2008

(54) Drilling microneedle device

(76) Inventors: Ping Ming Wang, Atlanta, GA (US); Mark R. Prausnitz, Atlanta, GA (US)

Correspondence Address: ROPES & GRAY LLP;PATENT DOCKETING 39/41, ONE INTERNATIONAL PLACE, BOSTON, MA, 02110-2624, US

(21) Appl. No.: 11/699,206

(22) Filed: Jan. 29, 2007

Related U.S. Application Data
(63) Continuation of application No. 10/861,244, filed on Jun. 4, 2004, now abandoned.
(60) Provisional application No. 60/476,015, filed on Jun. 4, 2003.

(51) Int. Cl. A61M 5/158

(52) U.S. Cl. 604/115; 604/117; 604/272; 606/180

(57) ABSTRACT

Rotating microneedles and microneedle arrays are disclosed that “drill” holes into a biological barrier, such as skin. The holes can of controlled depth and diameter and suitable for microsurgery, administering drugs and withdrawal of body fluids.

### REFERENCE TO RELATED APPLICATIONS

This application claims the benefit of the filing date of U.S. Provisional Application No. 60/476,015, filed on Jun. 4, 2003, the entire content of which is incorporated herein by reference.

### STATEMENT REGARDING FEDERALLY SPONSORED RESEARCH AND DEVELOPMENT

This invention was made with government support under Contract Number 1 R01 GM 60004-01A1, awarded by the National Institute of Health (NIH). The United States Government has certain rights in this invention.

### BACKGROUND OF THE INVENTION

The invention relates to injection/extraction devices, especially devices using a rotating microneedles, and to methods of using the same.

Delivery of drugs to a patient (e.g. human and other non-human animals) can be performed in a number of ways. For example, intravenous delivery is by injection drugs directly into a blood vessel of the patient; intraperitoneal delivery is by injection into the peritoneum; subcutaneous delivery is under the skin; intramuscular is into a muscle; and orally is through the mouth. One of the easiest methods for drug delivery, and for collection of body fluids, is through the skin. Recently, microneedles have been developed that penetrate the skin to a depth of less than 1 mm. The penetration depth of microneedles into the skin may be determined by many factors, such as the shape and diameter of the needle, the pressure/force applied to the needle, as well as other characteristic properties, such as the elasticity of the skin, and the needle-skin interaction (for example, the speed with which the needle is inserted into the skin). Certain conditions, such as diabetes and other chronic conditions, can be especially taxing because they require ongoing diagnostic and therapeutic intervention which may not only be inconvenient and/or painful, but also pose a serious risk of infection. It would therefore be desirable to provide an improved system and method for controllably puncture a tissue barrier for injecting/withdrawing materials (drug/gene/body fluids, etc.).

### SUMMARY OF THE INVENTION

The invention relates to methods and devices, and more particularly to microneedle devices with rotating or drilling microneedles, that improve and control the penetration of biological barriers (most commonly skin) for microsurgery, drug delivery, monitoring of, for example, glucose levels, intracellular gene transfer and the like.

According to one aspect of the invention, a microneedle or microneedle array is disclosed that can be used for transdermal penetration by rotating the microneedle(s). The microneedle, and particularly the tip of the microneedle, can have various shapes, for example, blunt, sharp, beveled, serrated, conical and/or frustoconical. The rotating microneedle operates much like a drill bit and can have a spiral-shaped material disposed on the outside surface of the microneedle tip to facilitate the drilling motion.

The rotating microneedle can include a plurality of rotating microneedles. The plurality of microneedles can either rotate together about a common axis, or each microneedle can be driven separately, for example, via a common drive shaft and suitable gearing, for example, a toothed gear. The toothed gear can be manufactured in a material suitable for micromachining, such as silicon.

The rotating microneedle can be fabricated of glass, silicon, metal, and can optionally be provided with a plastic coating to provide added rigidity to the needle(s). The materials used to construct the microneedle is preferably clear or transparent, at least translucent, so that position of the liquid within may be easily discerned.

The penetration depth of the microneedle can optionally be controlled by a variety of mechanisms. For example, in one embodiment, a limit stop may be placed in the applicator housing that cooperates with the propulsion mechanism of the microneedle for stopping the advance of the microneedle when the microneedle extend a certain distance from, for example, the surface of the applicator facing the skin. The insertion depth may be adjustable.

The surface of the skin to be penetrated can be “conditioned” to avoid skin-elastic effect and thereby better control the penetration depth by, for example, stretching the skin. This can be achieved by applying vacuum suction, by clamping the skin, or otherwise spreading/stretching the skin, for example, over rounded surface.

According to another aspect of the invention, a microneedle may be constructed so as to cooperate with a ballpoint pen-shaped applicator, which can be actuated by a spring activated by a push button. The microneedle is then pushed to puncture the skin. After the use, the microneedle may be released/retracted into the applicator, preferably through pushing the same push button. The applicator can also include a rounded surface or suction cup-shaped tip proximate to the microneedle, which aid in stretching the skin for controlled injection. The microneedle, in particular a microneedle made of glass, can be coated, for example, with plastic material so as to prevent injury to a patient in the event that the microneedle tip breaks when penetrating the skin.

Thus one aspect of the invention provides a microneedle device comprising: a microneedle tip for penetrating a biological barrier, said microneedle adapted to rotate about a longitudinal axis before, during, and/or after the penetration of the biological barrier.

In one embodiment, the microneedle device comprises: (1) a holder with a bottom surface for contacting said biological barrier, and an opening in said bottom surface allowing said microneedle to pass through; and (2) an insert rotatably disposed inside said holder, said insert having a through bore configured to receive said microneedle so positioned to pass through said opening.

In one embodiment, the bottom surface is convex.

In one embodiment, the bottom surface is concave.

In one embodiment, the concave-shaped bottom surface has a port connected to a suction device for applying a suction force and stretching said biological barrier.

In one embodiment, the bottom surface has a beveled-shape, a dome-shape, an inverse dome shape, a curve with the outside-shape of a barrel, a curve with the inside-shape of a barrel, or is connected to a suction cup.

In one embodiment, the biological barrier is skin.

In one embodiment, the outside surface of said insert engages the inside surface of said holder through spiral-shaped grooves or threads.

In one embodiment, the threads are on the outside surface of said insert.

In one embodiment, the maximum displacement distance of said insert relative to said holder along the longitudinal axis is limited by a limit stop protruding from the outside surface of said insert, at a pre-determined position from the top of said holder.

In one embodiment, the position of said limit stop is adjustable relative to the insert.

In one embodiment, the maximum displacement distance of said insert relative to said holder along the longitudinal axis is limited by a limit stop protruding from the inside surface of said holder, at a pre-determined position from the bottom of said insert.

In one embodiment, the position of said limit stop is adjustable relative to the holder.

In one embodiment, the outside surface of said insert engages the inside surface of said holder through spiral-shaped grooves or threads, and wherein the maximum displacement distance of said insert relative to said holder along the longitudinal axis is limited by a limited depth of said grooves or threads on the inside surface of said holder.

In one embodiment, the microneedle device further comprises a sealing element for sealing the space of the microneedle tip against the ambient.

In one embodiment, the microneedle device further comprises an O-ring between said sealing element and said insert, for sealing the microneedle against said insert.

In one embodiment, the movement of said insert along the longitudinal axis is effectuated by a mechanical coupling element attached to said insert.

In one embodiment, the mechanical coupling element comprises a wrench flat.

In one embodiment, the mechanical coupling element comprises a gear for coupling to another gear, a motor, or a micromotor.

In one embodiment, the mechanical coupling element comprises a handle.

In one embodiment, the microneedle device has an expanding spring for pushing the top of said insert.

In one embodiment, the microneedle device has a retracting spring inside said holder for pulling the bottom of said insert.

In one embodiment, the microneedle device has a vacuum for generating a sub-atmospheric pressure inside the chamber bounded by the bottom of the insert, the inside wall of the holder, and the portion of the biological barrier contacting the opening, and wherein said vacuum or sub-atmospheric pressure is generated by a suction device connected to said chamber.

In one embodiment, the microneedle device further comprises a spring inside said chamber, wherein the extension force generated by said spring facilitates retraction of said microneedle from said biological barrier after the vacuum is released.

In one embodiment, the microneedle is connected to a fluid reservoir storing fluids to be delivered across the biological barrier.

In one embodiment, the fluid reservoir generates a positive pressure to force the fluids into the microneedle.

In one embodiment, the positive pressure is generated after the penetration of said microneedle tip into the biological barrier.

In one embodiment, the microneedle is connected to a fluid reservoir for storing fluids extracted below the surface of the biological barrier.

In one embodiment, the fluid reservoir generates a negative pressure to extract fluids through the microneedle and from below the penetrated biological barrier.

In one embodiment, the negative pressure is generated after the penetration of said microneedle tip into the biological barrier.

In one embodiment, the microneedle tip is tapered.

In one embodiment, the microneedle tip is blunt.

In one embodiment, the microneedle tip is serrated.

In one embodiment, a spiral pattern is disposed on the outer surface of the microneedle tip.

In one embodiment, the microneedle tip is made of glass and covered with a plastic material.

In one embodiment, the microneedle tip is transparent/translucent.

In one embodiment, the microneedle device further includes a suction cup or mechanical stretching device to stretch the biological barrier to facilitate penetration by the microneedle tip.

In one embodiment, the insert comprises a plurality of through-bores, each configured to receive one additional microneedle, said microneedles are so arranged for rotating about a common longitudinal axis.

In one embodiment, the tips of said microneedles are so arranged to converge to the same area.

In one embodiment, each of said microneedles is independently connected to its own fluid reservoir.

In one embodiment, at least two of said fluid reservoirs contain different fluids.

In one embodiment, the insert comprises a plurality of through-bores, each configured to receive one additional microneedle, said microneedles are so arranged for rotating about their own longitudinal axis.

In one embodiment, the microneedle device further comprises a drive to commonly drive at least two of said microneedles.

In one embodiment, the drive includes a common drive shaft with a gear wheel that engages with gear wheels disposed on the commonly driven microneedles.

In one embodiment, the microneedle is made of glass, silicon, or metal.

In one embodiment, the microneedle is made of a transparent or translucent material.

In one embodiment, the microneedle is coated with a plastic or polymer layer.

In one embodiment, the maximum penetration depth into the biological barrier is less than 1 mm or 500 μm.

In all the embodiments described above, features of one embodiment can be freely combined with those of one or more other embodiments as appropriate.

Further features and advantages of the present invention will be apparent from the following description of preferred embodiments and from the claims.

### BRIEF DESCRIPTION OF THE DRAWINGS

The following figures depict certain illustrative embodiments of the invention in which like reference numerals refer to like elements. These depicted embodiments are to be understood as illustrative of the invention and not as limiting in any way.

FIG. 1 shows a cross-sectional views of a first embodiment of a drilling microneedle device.

FIG. 2 shows a cross-sectional views of a second embodiment of a drilling microneedle device with (a) applied pressure and (b) suction.

FIG. 3 shows a cross-sectional views of a third embodiment of a drilling microneedle device with a connected syringe and suction device.

FIG. 4 shows a beveled microneedle holder for drilling penetration.

FIG. 5 shows a microneedle device having multiple microneedles, with each microneedle rotating about its own axis.

FIG. 6 shows a microneedle device having multiple microneedles rotating about a common axis.

FIG. 7 shows a ballpoint-pen-shaped applicator with microneedle and suction cup.

FIG. 8A shows a flat-tipped hollow microneedle for drilling penetration.

FIG. 8B shows a serrated-tipped hollow microneedle for drilling penetration.

FIG. 8C shows a tapered-tipped hollow microneedle for drilling penetration.

FIG. 8D shows a spiral-tipped hollow microneedle for drilling penetration.

FIG. 9 shows a cross-sectional views of a fourth embodiment enabling simultaneous advance and rotation of the microneedle.

FIG. 10 shows the advance of the tip of, the microneedle device of FIG. 9 with rotation.

FIG. 11 shows a system with separate position and depth control for depth-controlled drilling with microneedles.

FIG. 12 shows a top view and diameter versus depth of a hole drilled into hairless rat skin.

FIG. 13 shows series of cross-section views of a drilling hole in a Z-directional scan. The lower panels show the diameters of the holes at the respective sections, and the corresponding drilling depths.

FIG. 14 shows a drilling hole generated by the subject microneedle device on hairless rat skin.

FIG. 15 is a cross-section image of hairless rat skin showing the diameter and depth of the hole shown in FIG. 14.

FIG. 16 shows the site of drilling penetration (top panel) and the deepest reach (379 μm) of the tissue blue marker prepared as 20% PBS solution and injected for 5 minutes under 10 psi.

FIG. 17 shows the cross-section of an extraction site.

FIG. 18 shows a flat glass hollow microneedle with a length of about 650 μm and a tip diameter of about 73 μm.

FIG. 19 shows several shapes of tips for the subject microneedles useful for drilling and/or extraction. The top left panel shows one with a tapered tip; the top right panel shows one with a flat tip.

FIG. 20 shows dimensions of an exemplary construction of the subject microneedle device.

FIG. 21 shows several views of a manufactured model of an exemplary embodiment of the subject microneedle device.

FIG. 22 shows a configuration of the exemplary embodiment in FIG. 21, with the microneedle coupled to a syringe as a fluid reservoir.

### DETAILED DESCRIPTION OF ILLUSTRATED EMBODIMENTS

The devices and methods described herein are directed, inert alia, to microneedles that facilitate penetration of a biological barrier (most commonly skin) of a human or non-human animal. More particularly, the subject devices and methods are directed to rotating microneedles and arrays of microneedle that puncture the skin by “drilling” holes. Such devices and methods are suitable for microsurgery, administering drugs and withdrawal of body fluids.

One salient feature of the subject microneedle device is the ability of one or more microneedles to rotate along a longitudinal axis while bearing down towards the biological barrier to be penetrated. Such rotary motion facilitates a smooth, steady, and controlled opening of a hole on the surface of the biological barrier. Thus the microneedle device operates much like a drill bit or a screw, instead of a nail abruptly penetrating a surface. Either during or after the drilling and penetration of the biological barrier, fluid can be either injected into or withdrawn from under the surface of the biological barrier, through the microneedle(s).

To facilitate the drilling motion, the microneedle(s) may be housed inside other structures, each with distinct functions. The following descriptions are merely several illustrative embodiments that are not intended to be limiting in any respect. A skilled artisan could readily conceive other similar embodiments without departing from the spirit of the invention.

In a general sense, the subject microneedle device may comprise (1) a holder with a bottom surface for contacting the biological barrier, and an opening in the bottom surface allowing the microneedle to pass through; and (2) an insert rotatably disposed inside said holder, said insert having a through bore configured to receive said microneedle so positioned to pass through said opening.

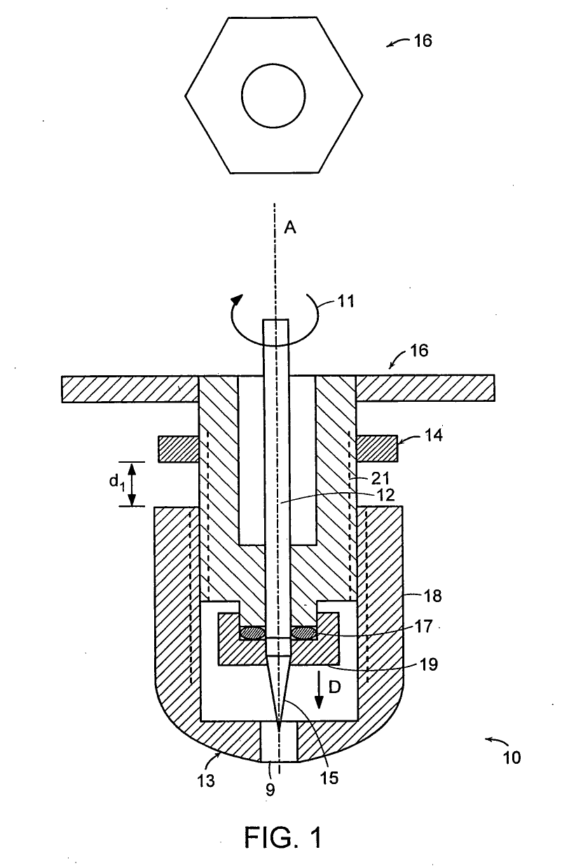

FIG. 1 shows a high level exemplary embodiment of such a rotating microneedle device 10 with a holder 18 having a bottom surface 13 adapted to contact a biological barrier, such as skin, and an insert 21 placed inside the holder 18. Insert 21 is rotatably disposed inside the holder 18. The insert 21 has a through bore configured to receive a microneedle 12 which has a tip 15 adapted to project through an opening 9 disposed in the bottom surface 13 of the holder 18. The insert 21 with the microneedle 12 can rotate in the holder 18 about its longitudinal axis A, as indicated by arrow 11, and can also be displaced along the axis A, as indicated by arrow D. The longitudinal displacement along D is constrained by a maximum distance d₁ by a limit stop 14 disposed on the insert 21. An optional sealing element 19 seals the space of the needle tip 15 against the ambient, with an optional O-ring 17 sealing the needle 12 against the insert 21. To facilitate rotation of the insert 21 and hence also the needle 12 relative to the holder 18, a wrench flat or another type of mechanical coupling element 16 can be formed on or attached to the insert 21.

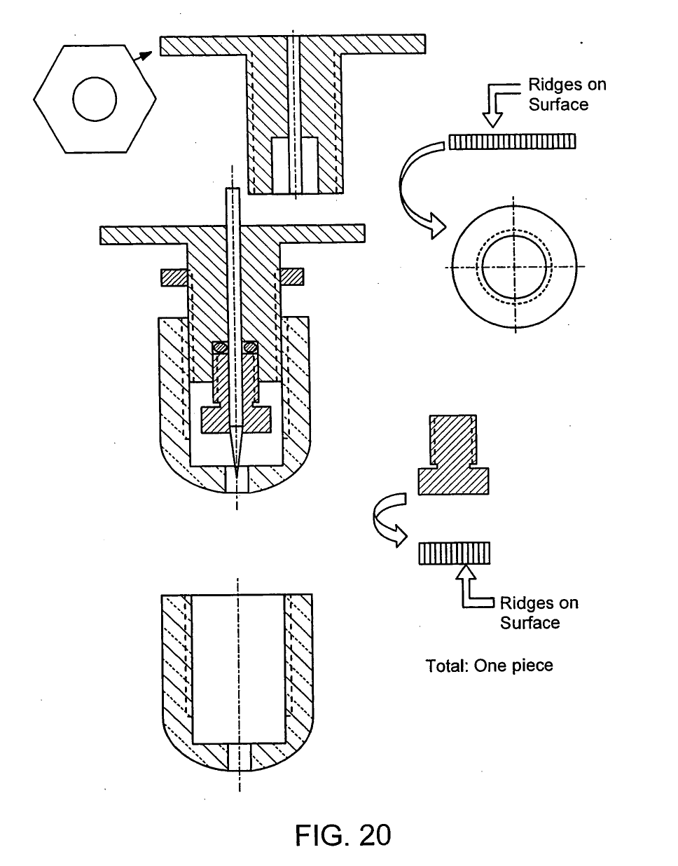

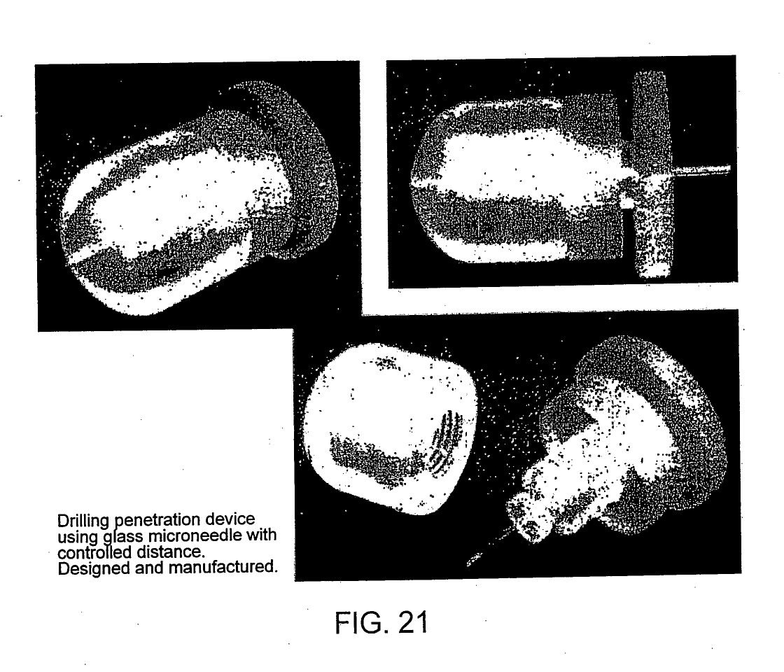

FIG. 20 shows the dimensions of an exemplary construction of one embodiment of the subject microneedle device. All measures are in inches, and are subject to variation (both proportional and disproportional) based on specific needs. FIG. 21 shows several views of an actual model of one embodiment of the subject microneedle device. The top left panel shows the holder and the insert as a single piece. The sides of the limit stop and the wrench flat have rough surfaces to facilitate manual operation (rotation). A tiny tip of the microneedle is also shown emerging from the center of the convex bottom surface. Note that the maximum penetration depth of most microneedles are less than 1 mm. Top right panel shows the side view of the same device. The bottom panel shows the holder and the insert (with microneedle) as two separate pieces. The groove on the inside wall of the holder is also visible. The insert has the optional sealing element in this particular embodiment.

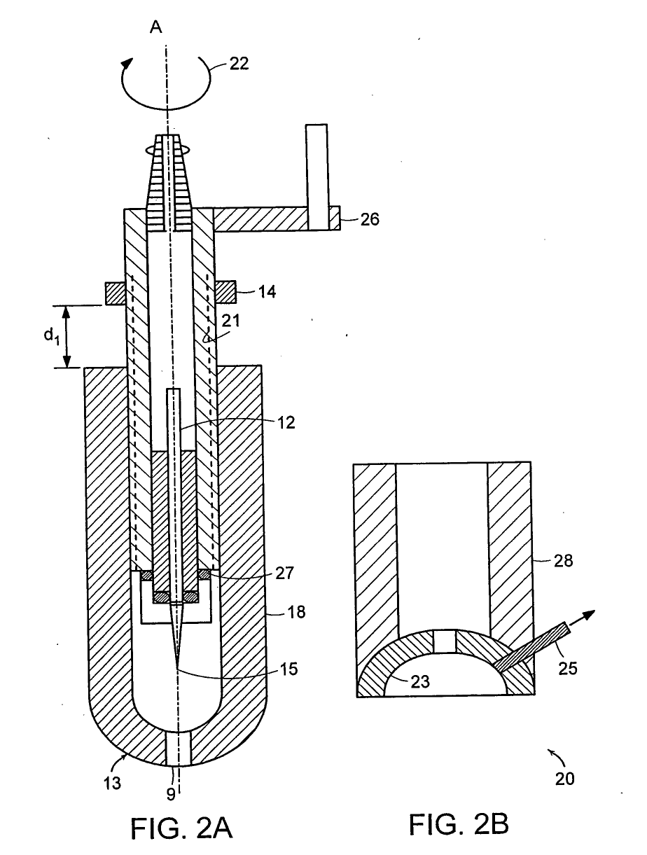

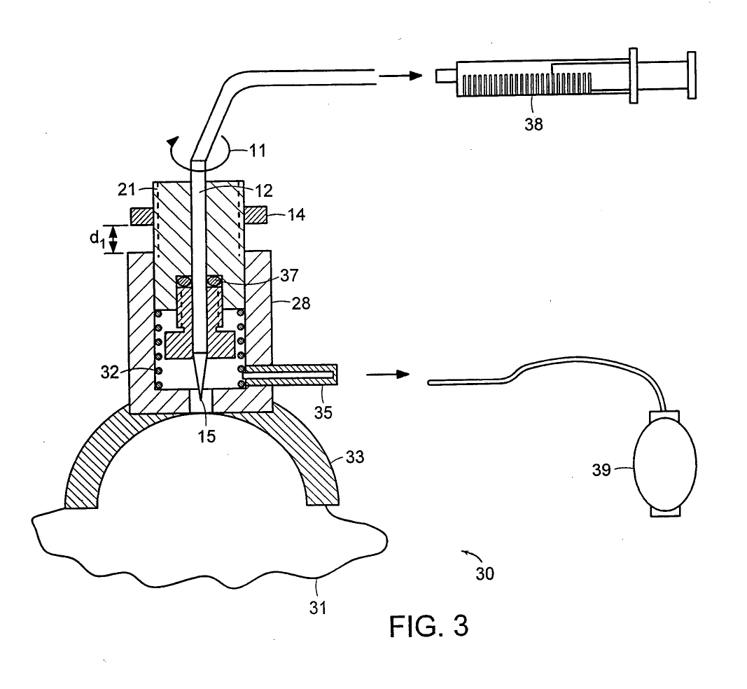

In one embodiment, the bottom surface of the holder is shaped in such a way to “condition” the surface of the biological barrier so as to eliminate/reduce the elastic effect of the biological barrier. There could be many different shapes of the bottom surface to stretch, for example, the skin to achieve this effect. In one preferred embodiment, the bottom surface is convex or concave, such that the surface of the biological barrier is stretched when the convex or concave bottom surface is pressed against the biological barrier. For a concave-shaped bottom surface, a port on the bottom surface may be used to connect to a suction device, so that a tighter fit between the biological barrier and the bottom surface can be achieved. See FIGS. 2(b) and 3.

Alternatively, the bottom surface may have a beveled-shape, a dome-shape (concave), an inverse dome shape (convex), a curve with the outside-shape of a barrel, a curve with the inside-shape of a barrel, etc., or is directly connected to a suction cup. In case of a suction cup, which can be made of medical rubber, pressing the cup squeezes out air and creates a negative pressure inside the suction cup, which helps to pull the skin surface taut (see FIG. 7).

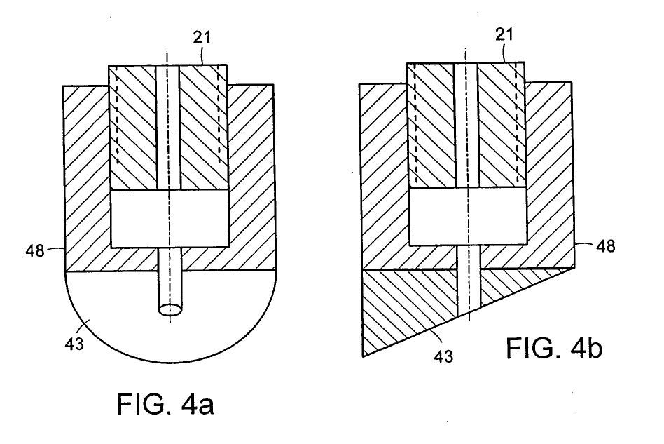

As shown in FIG. 4, the skin (not shown in FIG. 4) can also be stretched by providing the bottom of the holder 48 with a beveled surface 43. FIG. 4(a) shows a front cross-sectional view of the holder 48, while FIG. 4(b) shows a side cross-sectional view of the same holder. The inserts and microneedles can be constructed as in the afore-described embodiments.

Although in theory, the subject microneedle device can be applied to any kind of biological barrier, the most common type of biological barrier is skin. In certain embodiments, to avoid potential interference, hairs on the skin area to be contacted with the bottom surface of the holder may be partially or completely removed by, for example, shaving the surface of the skin.

The insert may move longitudinally inside the holder through a variety of means. The insert itself does not necessarily rotate, so long as the microneedle inserted therein can (see below). But in certain embodiments, when the microneedle is affixed to the insert (immobile relative to the insert), the insert itself may rotate.

In one embodiment, the rotation movement of the insert and its longitudinal movement inside the holder are uncoupled. For example, the rotation may be generated by rotating the insert while simultaneously applying a downward force towards the bottom of the holder. Such longitudinal movement is relatively unguided, depending largely on the amount of forces applied.

In another embodiment, the rotation movement of the insert and its longitudinal movement inside the holder are coupled, through, for example, the use of spiral-shaped grooves or threads on the surfaces of the insert and the holder. For example, in one embodiment, the outside surface of the insert has threads that fit into the grooves on the inside wall of the holder. When the insert is forced towards the bottom of the holder, it is also forced to rotate either clockwise or counter-clockwise, depending on the orientation of the grooves. In an opposite arrangement, the grooves are on the outside surface of the insert, while the threads are on the inside surface of the holder.

To control the maximum displacement distance of the insert inside the holder, or the maximum penetration depth by the microneedle into the biological barrier, several mechanisms may be employed to stop the longitudinal movement of the insert after certain pre-determined displacement distance has been reached.

In one embodiment, as shown in FIG. 1, a limit stop may be affixed to the upper portion of the insert, so that the limit stop will eventually clash with the top portion of the holder and prevent further longitudinal displacement of the insert. The limit stop need not be a continuous circle, as suggested in FIG. 1, so long as it protrudes from the surface of the insert in such a way to prevent it from going deeper into the holder. For a circular shaped limit stop, it can also be used as a dial to rotate the insert. In the latter case, the side of the limit stop may have a rough surface (such as a scored or threaded surface) to facilitate tighter finger grip or coupling to mechanical rotating devices).

In another embodiment, the limit stop may be situated inside the holder (such as a ring or a bump on the inner wall of the holder) to prevent further advancement of the insert when the insert reaches the limit stop.

In these embodiments, the position of the limit stop may be adjustable to allow different penetration depth, which is preferably less than about 1 mm, or less than about 800 μm, or about 500 μm, or about 400 μm, or about 300 μm, or about 200 μm, or about 100 μm, or about 50 μm.

In yet another embodiment, if the insert and the holder is coupled through thread and groove, the termination of the groove pattern on the inner wall of the holder will effectively stop the longitudinal movement of the threaded-insert.

The movement of the insert can be effectuated by a number of means. Without limitation, such means may range from simple manual pushing to mechanized pushing and/or rotating the insert.

In one embodiment, the top of the insert may be attached to a wrench flat (as shown in FIG. 1) or other mechanical coupling elements. The wrench flat can be any shape, such as a hexagon, so long as it can be easily used to rotate the insert. Again, a scored or rough surface at the side of the wrench flat may facilitate easy rotating.

Alternatively, as shown in FIG. 2(a), a handle or level may be used to rotate the insert. FIG. 2(a) shows a second exemplary embodiment of a rotating microneedle device 20, which is similar to the embodiment depicted in FIG. 1, with the exception that the rotation is accomplished by using a handle or crank. The bottom surface 13 which helps to stretch the skin is formed convex. FIG. 2(b), on the other hand, has a holder 28 with a concave bottom surface 23 with a port 25 to which a suction device (not shown in FIG. 2; see FIG. 3) can be connected. When suction is applied to the port, the skin is being stretched.

In another related embodiment, the insert can be rotated by attaching it to a gear, a motor or micromotor, or any other mechanical device that can rotate the insert. The motor may be programmed to rotate the insert at a pre-determined speed, either constant or changing according to a scheme (slower first, then faster, etc.), over a predetermined period of time (e.g. 5 minutes, 10 minutes, 15 minutes etc.).

In still another embodiment, spring mechanism may be employed to push the insert. In one variation, an extending spring force may be applied at the top of the insert to push it down into the holder. The rotation may be generated, in this situation, by using grooves and threads described above. In another variation, a retraction/pulling spring force may be applied at the bottom of the insert to pull it towards the bottom of the holder.

In still another embodiment, as illustrated in FIG. 3, a vacuum or sub-atmospheric pressure may be generated inside the chamber bounded by the bottom of the insert, the inside wall of the holder, and the portion of the biological barrier contacting the opening. The vacuum or sub-atmospheric pressure may be generated by a suction device connected to said chamber. Such a situation is shown in FIG. 3, which is another exemplary embodiment of a rotating microneedle device 30 with a connected syringe 38 adapted to supply a drug and/or withdraw body fluids through the microneedle 12. Also shown is a vacuum bulb 39 to apply a vacuum to the space enclosed by the bottom surface 33 and the skin 31. As also shown in FIG. 3, a spring 32 can be placed between the holder 28 and the insert 21 which facilitates retraction of the microneedle 12 from the skin 31. It will be understood from FIG. 3, that suction can also be used to propel the microneedle tip 15 against the skin 31.

The microneedle may be attached to the insert by any suitable means. In one embodiment, the microneedle is fixed onto the insert and is thus immobile relative to the insert. In this configuration, if a single microneedle is used, the microneedle and the insert preferably share the same rotating axis. Alternatively, if the microneedle is not located in the center of the insert, the tip of the microneedle may move in a circular motion and scratch the surface of the biological barrier, a desirable situation in certain situations.

In another related embodiment, the microneedle is not fixed relative to the insert (movable relative to the insert). This is most useful if the microneedle is driven by its own rotating force (such as by an attached micromotor), and the insert is driven down by another force towards the bottom of the holder. In that configuration, the insert do not need to be rotated itself, and it can move straight down, with or without the help of a guide on the wall of the holder, such as a groove. Also in that configuration, the microneedle needs not to be at the center of the insert.

The microneedle may be connected to a reservoir. In one embodiment, the reservoir is a storage tank for fluids to be delivered across the biological barrier. In this embodiment, the stored fluids may be forced into the microneedle under a positive pressure, preferably after the microneedle has penetrated into the biological barrier.

In another embodiment, the microneedle is connected to a reservoir that serves a storage tank for liquids/fluids extracted through the microneedle. In that embodiment, the reservoir may be connected to a vacuum source so that the fluids can be extracted through the microneedle under a negative pressure. To prevent clogging the microneedle tip, a positive pressure may be maintained during the drilling of the biological barrier, and a negative pressure is applied once the drilling is complete and extraction of fluid begins.

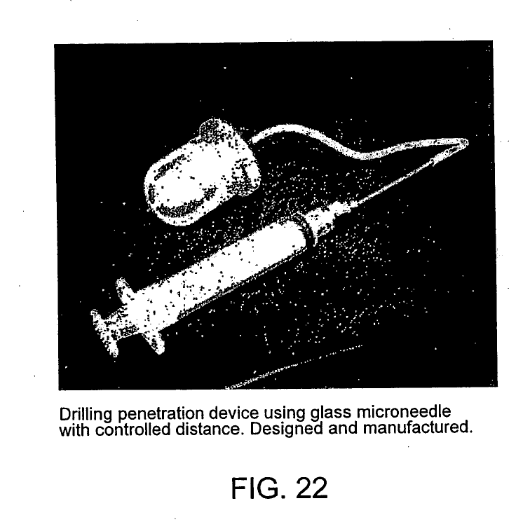

FIG. 22 shows an exemplary embodiment where a subject microneedle device is attached to a syringe serving as a fluid reservoir. In this configuration, the syringe can either be a storage tank for fluids to be injected through the microneedle, or be a collection device for fluids extracted from under the biological barrier after the penetration of the barrier by the microneedle device.

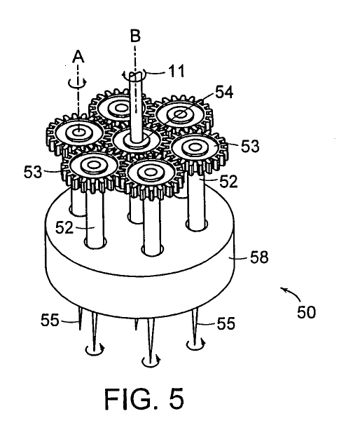

Having only a single microneedle secured in a holder is limiting for practical applications. For example, the small inside diameter of the microneedle allows only a certain flow rate of the drug and/or fluid to be supplied/withdrawn through the microneedle. In addition, simultaneous delivery of multiple drugs may be difficult or impossible. These disadvantages can be overcome by arranging a plurality of microneedles on a holder 58, as depicted in FIG. 5. FIG. 5 shows an arrangement of microneedles 52 arranged concentrically around an axis B. The microneedles can rotate separately about their respective axes A. The microneedles can be geared to a common drive shaft 54 which is aligned with the axis B. When the drive shaft 54 rotates in a direction 11, all microneedles 52 rotate with an opposite rotation sense, causing each tip 55 to piece the skin at a different location. The assembly 50 can be combined with any one of the afore-described holders, and the microneedles 52 can be connected to different drug reservoirs or to a common reservoir.

In a variation embodiment, not all microneedles in the array is concentrically arranged, and not all microneedles are coupled to the same drive shaft. For example, a cluster of microneedles may be centered around one common drive shaft, while another cluster of microneedles may be centered around another common drive shaft, such that the rotation of the two clusters can be separately regulated. In addition, some of the microneedles in the microneedle array may not be engaged with any drive shafts, and instead can be driven individually if desired. In these embodiments, the longitudinal movement of the insert and the rotation of the microneedles are preferably uncoupled.

In one embodiment, each microneedle is attached to a separate reservoir, which may contain the same or different fluids that can be independently delivered through the biological barrier at the same or different time points.

Due to their small size, the gears for the microneedles may advantageously be machined by micro-machining techniques, for example, from silicon.

The pattern in which the microneedles are arranged and the geared drive mechanism are exemplary only, and are not limited to the illustrated versions. Other arrangements and mechanisms known in the art can be readily used as long as at least some of the microneedles can be individually driven. A common driveshaft 54 is also not required, as the microneedles could be driven by miniature electric motors or by pneumatic and/or hydraulic actuators.

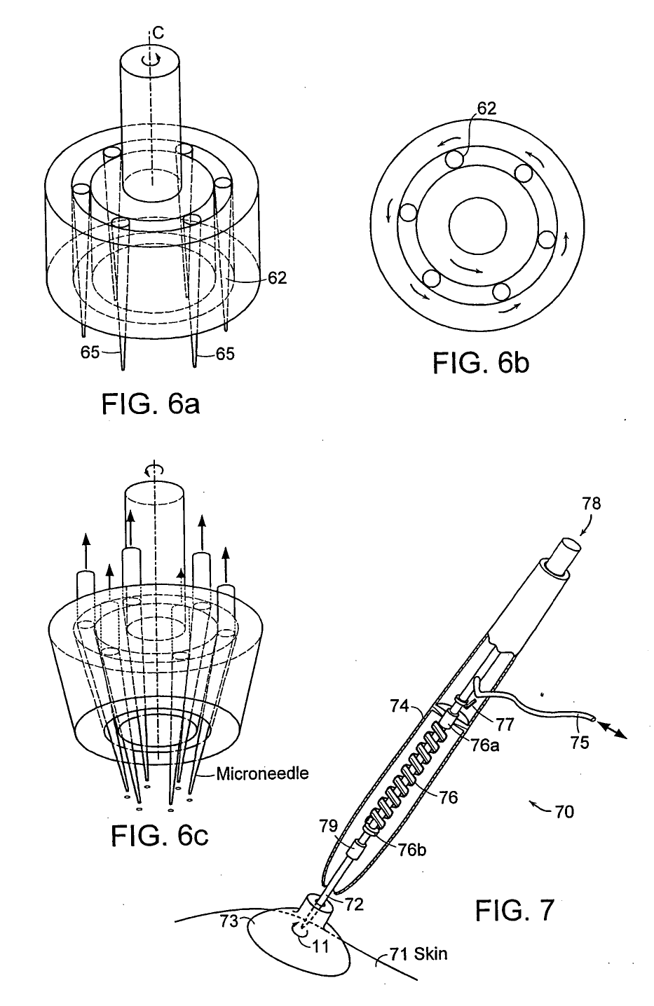

FIGS. 6(a) and (b) show in a perspective view and in a top view another embodiment where exemplary microneedles 62 are fixedly arranged on a common insert that can rotate about a rotation axis C. The microneedles 62 would here “scratch” instead of puncture the skin. In another modified embodiment depicted in FIG. 6(c), the microneedles 62 can be arranged so that the tips 65 converge to almost a point, which would produce a controlled skin puncture with a smaller diameter, while allowing simultaneous administration of drugs from multiple reservoirs.

FIG. 7 shows a ballpoint pen-shaped spring-loaded applicator 70 with a housing 74 and a suction cup 73 disposed at the tip of the housing 74 and contacting the skin 71. A microneedle device 72 is attached to a piston-like arrangement 79 supported by the housing. A spring 76 applies a spring force between a support collar 76a affixed to the housing 74 and another collar 76b on the piston. In a retracted position, the needle is held under spring force against the housing by a catch 77. When an operator clicks a button 78, the catch disengages from the piston and the microneedle 72 is propelled against the skin 71. The piston 79 can also cooperate with the interior lumen of the hollow microneedle 72 and can be connected to a catheter to either supply a drug or withdraw body fluid by suction, as described above with reference to, for example, FIG. 3. The housing 74 and/or piston assembly 79 can also be configured to apply suction to the tip 73.

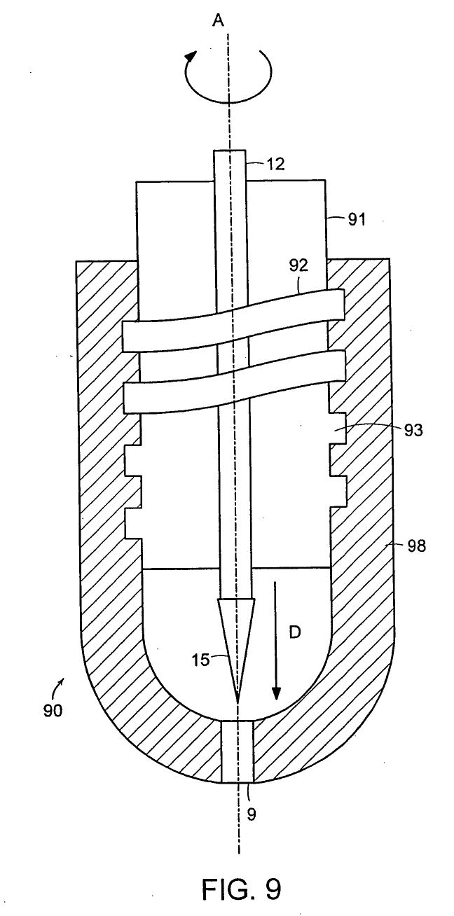

Advantageously, the piston assembly 79 may also include spiral grooves that engage with complementary grooves in the housing 74 (see, for example, FIG. 9). In this way, the microneedle 72 will rotate about the longitudinal axis of the housing 74 when the microneedle 72 is propelled against the skin, resulting in the afore-described advantageous drilling motion of the microneedle.

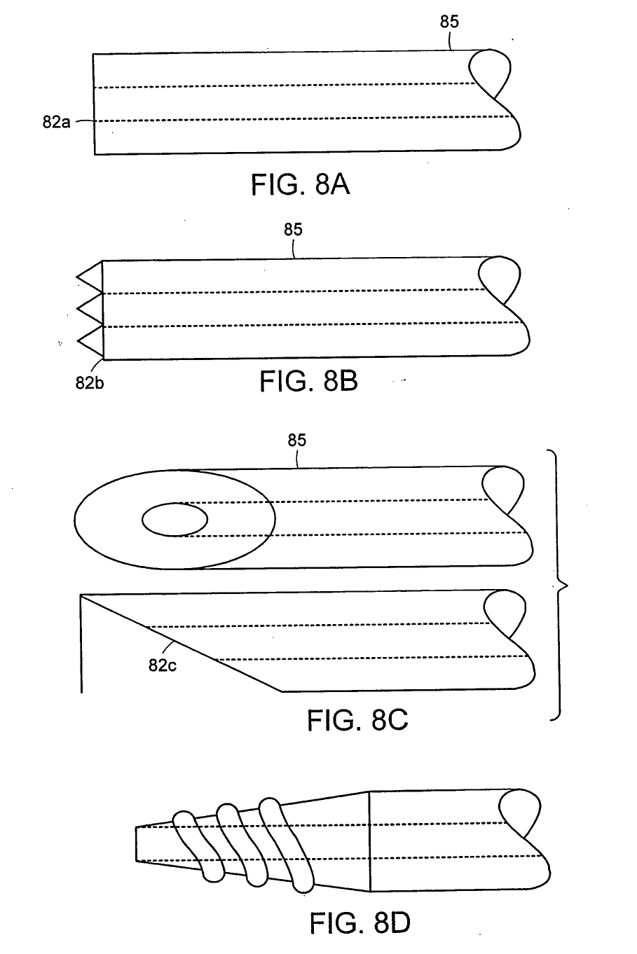

The tips of the microneedle(s) may take various shapes. FIGS. 8(A)-8(D) depict several exemplary shapes of microneedle tips for the subject rotating microneedles. Tip 82a of microneedle 85 is blunt, but still performs adequately when used with a rotating microneedle. A better performance can be obtained with either a serrated tip 82b (FIG. 8B) or a tapered tip 82c (shown in two sectional views in FIG. 8C). The microneedle tip can also have a spiral disposed on the outside surface of the tip (FIG. 8D), in which case the microneedle operates more like a drill bit.

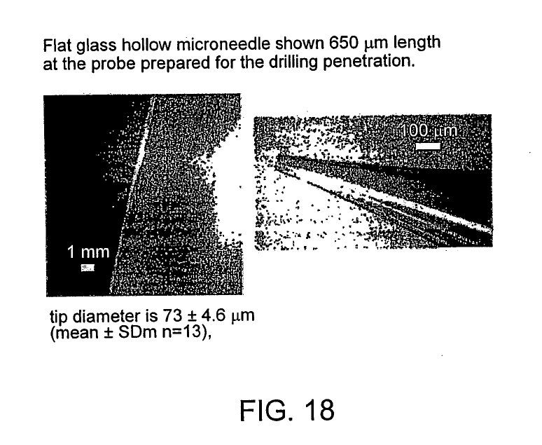

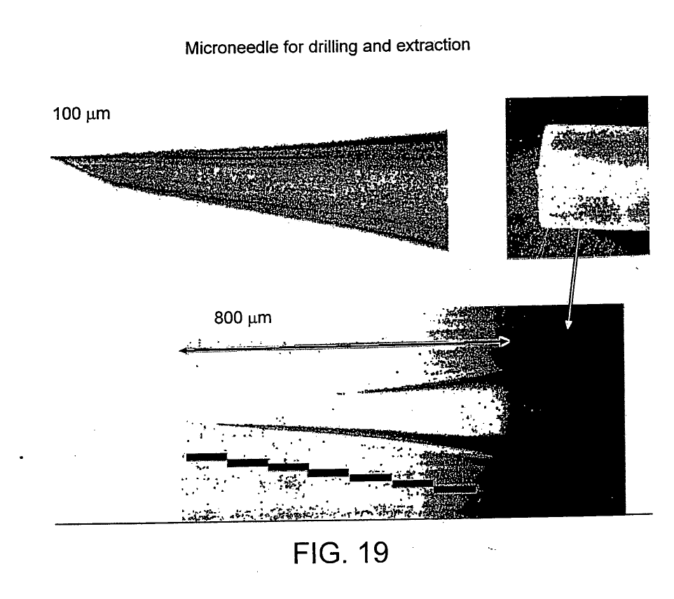

FIGS. 18 and 19 show several manufactured exemplary embodiments of microneedles with different kinds of tips (tapered with a beveled opening at the tip; flat; tapered with a flat tip, etc.)

While in some of the microneedle devices described above, the rotation of the microneedles is uncoupled from the movement of the microneedles against the skin, the microneedle device 90 depicted in FIG. 9 pushes the microneedle tip 15 through the opening 9 against the skin when the insert 91 is rotated relative to the holder 98. This can be accomplished by providing the insert 91 with an exterior thread 92 which engages with grooves 93 disposed in the holder 98. It will be understood that the placement of thread and grooves can also be interchanged.

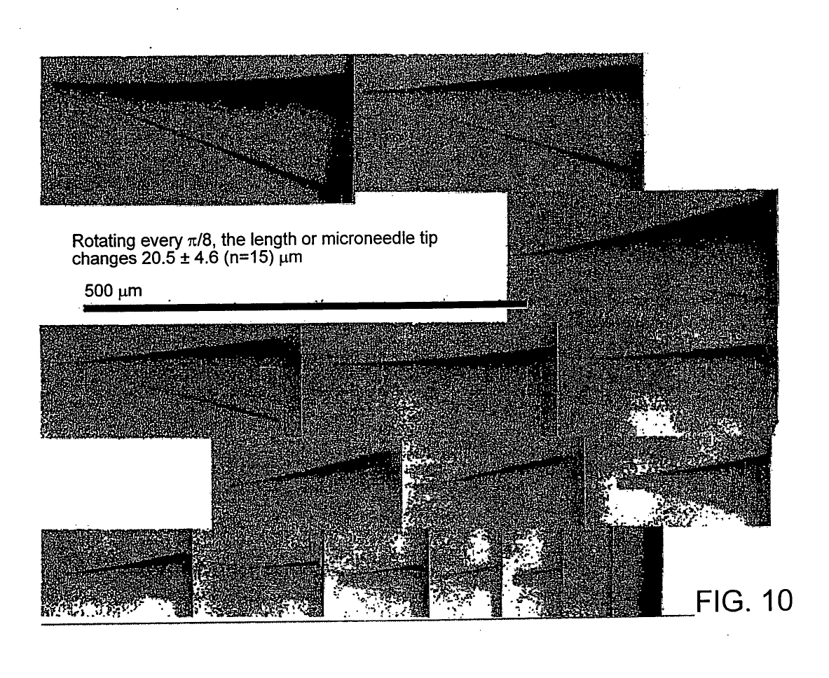

FIG. 10 is an image of the microneedle tip which projects a successively greater distance out of the hole 9 when the microneedle is rotated. In the illustrated embodiment, the length of the microneedle tip changes by approximately 20.5 μm for each 22.5° rotation ( 1/16 of a turn) of the microneedle in the holder, for a total length change of approximately 330 μm per turn. Obviously, other values may be readily obtained, for example, by changing the pitch of the groove/thread on the wall of the holder/insert.

Drilling microneedles can also be used for micro-surgery, for example, eye surgery, eye drug delivery, gene transfer in developmental biology, for vascular studies, genetic studies, such as the penetration of cell walls of eggs and embryos, and other small tissue applications.

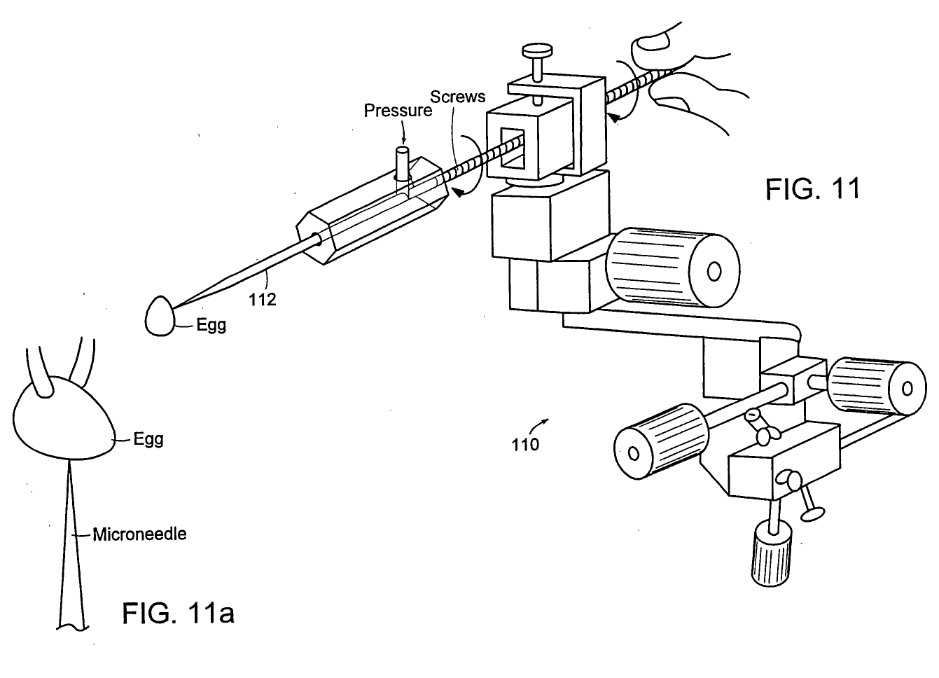

For such applications, a microneedle device 112 can be mounted on a conventional XYZ-stage 110 for position and depth control, as shown in FIG. 11.

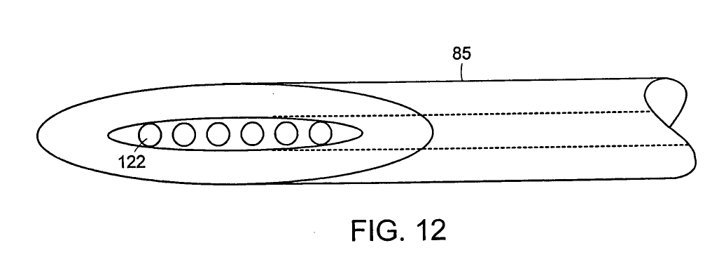

Because of the precise insertion and depth control that can be achieved with rotating microneedles, gene or antibody sensitive dots, for example, in form of microchips or “gene” chips, can be applied proximate to the microneedle tip. These dots are shown schematically in FIG. 12 and can be used in-vivo or in-vitro for the analysis of body fluids and other samples.

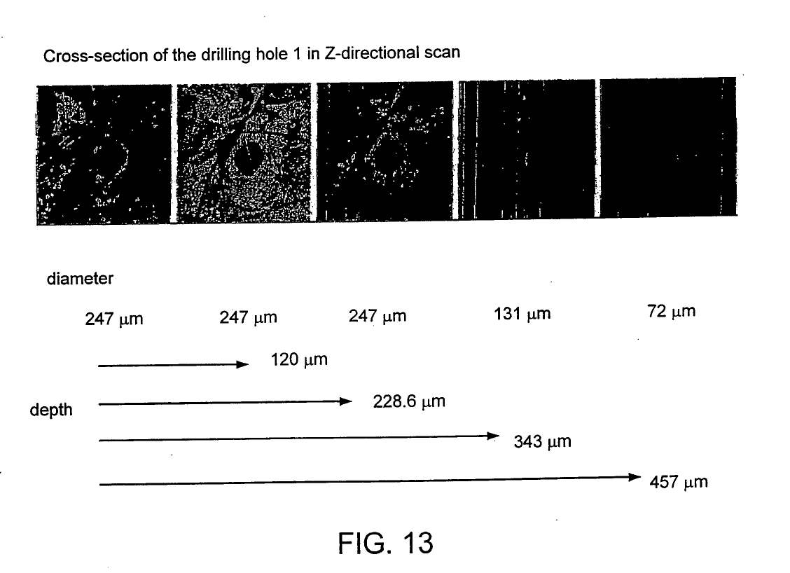

FIGS. 13 shows a small hole of controlled diameter and depth “drilled” with a rotating microneedle into hairless rat skin. The exemplary drilled hole has a diameter ranging from approximately 70 μm at a depth of up to approximately 450 μm, to approximately 250 μm in diameter when close to the surface. Obviously, the specific values may depend on the microneedle configuration, such as the needle taper, the set depth, the applied pressure, the skin stretching, etc. These values can be designed to adapt to specific applications.

The drilling method and microneedle drilling device have applications in many areas of biomedical research, pharmacotherapy, agriculture and the pharmaceutical industry, and more particularly in skin and other soft tissue drug delivery, transdermal interstitial fluid extraction, intracellular gene transplant, cytoplasmic injection to introduce purified DNA into fertilized eggs, vaccine delivery, cellular signal recording, gene transplant in the embryo, artificial insemination in eggs, acupuncture, and intravascular fluorescent dye or marker loading. The device and method can also be applied to plants.

Moreover, the puncture depth can be accurately preset and/or controlled by providing a stop ring whose position can be adjusted, for example, by using a (micrometer) screw arrangement.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Drilling Hairless-Skin with Microneedle Device

An area of rat skin was shaved to remove hair and reveal the skin surface underneath. A microneedle device as depicted in Figure xxx was used to drill holes on the hairless skin area, using a microneedle with a maximum drilling depth of about 800 μm.

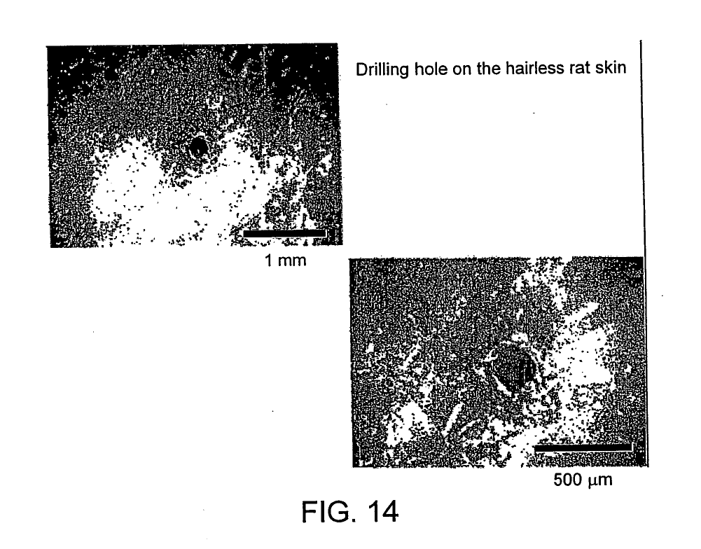

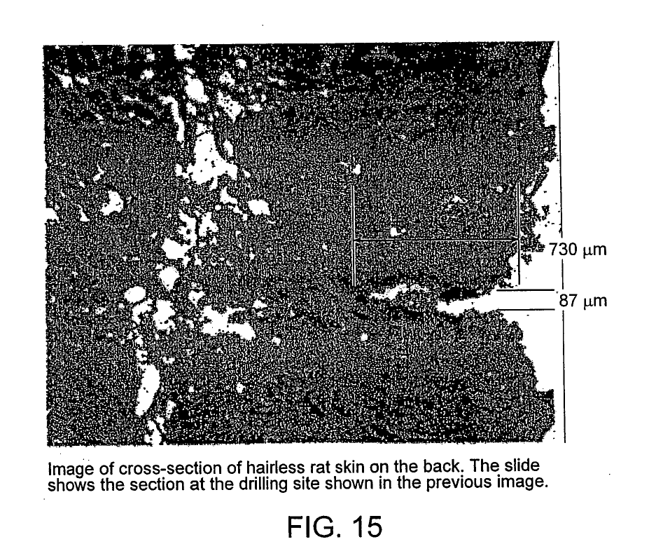

FIG. 14 shows, at two different magnification, that a single hole with a relatively round shape was generated after drilling. FIG. 15 is a cross-section of the hole shown in FIG. 14, obtained by freezing the drilled hole and sectioning using microtome. The figure shows that the drilling left in the skin a hole with a depth of about 730 μm, and a diameter of about 87 μm at the surface of the skin.

### Example 2

### Drilling Hairless-Skin with Microneedle Device, and ISF Collection

An area of bare skin was prepared as above. After drilling 3-10 points in the general area, a vacuum pressure of about −200 to −500 mmHg was applied to the area with drilled holes, for about 5-10 minutes. After suction, small interstitial fluids (ISF) and blood droplets appeared at the skin surface. The ISF collected through the vacuum, which was about 700 nL total in volume, turned out to be sufficient for glucose level monitoring using a standard glucose monitoring device, such as the FreeStyle™ blood glucose sensor (TheraSense, Alameda, Calif.). The measured glucose level is identical to the blood glucose level.

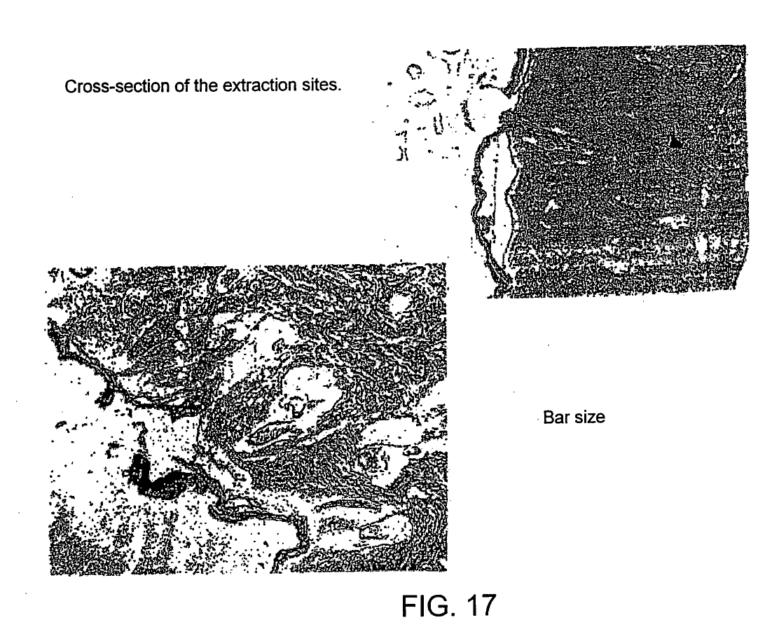

FIG. 17 shows a cross-section of the bare rat skin drilled for fluid extraction.

### Example 3

### Drilling Hairless-Skin with Microneedle Device and Fluid Microinjection

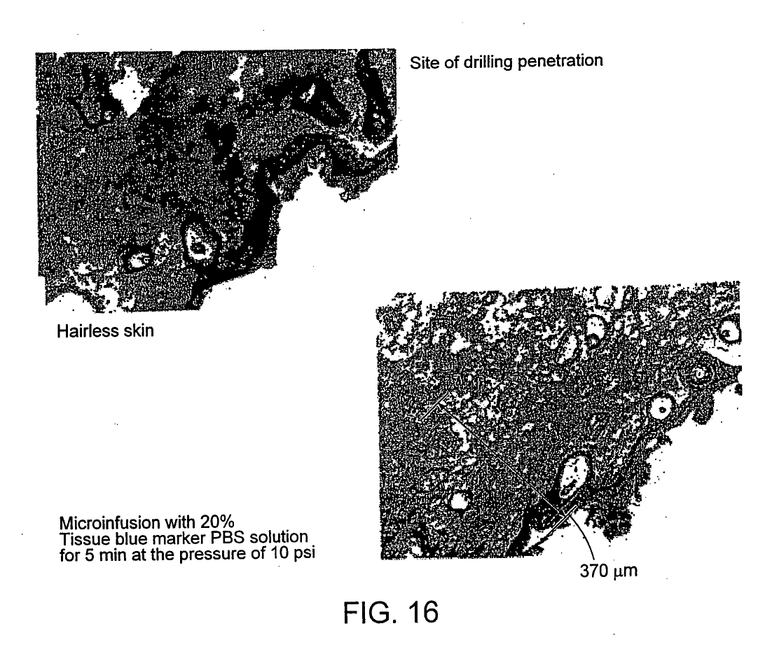

An area of bare skin was prepared as above. Tissue blue dye (marker) was prepared as 20% solution in PBS, and infusion of the dye solution through the subject microneedle device lasted about 5 minutes under a positive pressure of about 10 psi. The injected skin specimen was cut off and frozen in liquid N₂, and then sectioned using microtome to reveal the depth the dye reached. FIG. 16 shows that the deepest reach of the dye was about 370 μm, indicating that the subject device can be used to control the distance of needle reach, such that an automatic drug injection with a pre-determined depth can be achieved.

Equivalents

While the invention has been disclosed in connection with the preferred embodiments shown and described in detail, various modifications and improvements thereon will become readily apparent to those skilled in the art.

1. A microneedle device comprising: a microneedle tip for penetrating a biological barrier, said microneedle adapted to rotate about a longitudinal axis before, during, and/or after the penetration of the biological barrier.

2. The microneedle device of claim 1, comprising:
(1) a holder with a bottom surface for contacting said biological barrier, and an opening in said bottom surface allowing said microneedle to pass through;
(2) an insert rotatably disposed inside said holder, said insert having a through bore configured to receive said microneedle so positioned to pass through said opening.

3. The microneedle device of claim 2, wherein said bottom surface is convex.

4. The microneedle device of claim 2, wherein said bottom surface is concave.

5. The microneedle device of claim 4, wherein said concave-shaped bottom surface has a port connected to a suction device for applying a suction force and stretching said biological barrier.

6. The microneedle device of claim 2, wherein said bottom surface has a beveled-shape, a dome-shape, an inverse dome shape, a curve with the outside-shape of a barrel, a curve with the inside-shape of a barrel, or is connected to a suction cup.

7. The microneedle device of claim 2, wherein said biological barrier is skin.

8. The microneedle device of claim 2, wherein the outside surface of said insert engages the inside surface of said holder through spiral-shaped grooves or threads.

9. The microneedle device of claim 8, wherein said threads are on the outside surface of said insert.

10. The microneedle device of claim 2, wherein the maximum displacement distance of said insert relative to said holder along the longitudinal axis is limited by a limit stop protruding from the outside surface of said insert, at a pre-determined position from the top of said holder.

11. The microneedle device of claim 10, wherein the position of said limit stop is adjustable relative to the insert.

12. The microneedle device of claim 2, wherein the maximum displacement distance of said insert relative to said holder along the longitudinal axis is limited by a limit stop protruding from the inside surface of said holder, at a pre-determined position from the bottom of said insert.

13. The microneedle device of claim 12, wherein the position of said limit stop is adjustable relative to the holder.

14. The microneedle device of claim 2, wherein the outside surface of said insert engages the inside surface of said holder through spiral-shaped grooves or threads, and wherein the maximum displacement distance of said insert relative to said holder along the longitudinal axis is limited by a limited depth of said grooves or threads on the inside surface of said holder.

15. The microneedle device of claim 2, further comprising a sealing element for sealing the space of the microneedle tip against the ambient.

16. The microneedle device of claim 15, further comprising an O-ring between said sealing element and said insert, for sealing the microneedle against said insert.

17. The microneedle device of claim 2, wherein the movement of said insert along the longitudinal axis is effectuated by a mechanical coupling element attached to said insert.

18. The microneedle device of claim 17, wherein said mechanical coupling element comprises a wrench flat.

19. The microneedle device of claim 17, wherein said mechanical coupling element comprises a gear for coupling to another gear, a motor, or a micromotor.

20. The microneedle device of claim 17, wherein said mechanical coupling element comprises a handle.

21. The microneedle device of claim 2, having an expanding spring for pushing the top of said insert.

22. The microneedle device of claim 2, having a retracting spring inside said holder for pulling the bottom of said insert.

23. The microneedle device of claim 2, having a vacuum for generating a sub-atmospheric pressure inside the chamber bounded by the bottom of the insert, the inside wall of the holder, and the portion of the biological barrier contacting the opening, and wherein said vacuum or sub-atmospheric pressure is generated by a suction device connected to said chamber.

24. The microneedle device of claim 23, further comprising a spring inside said chamber, wherein the extension force generated by said spring facilitates retraction of said microneedle from said biological barrier after the vacuum is released.

25. The microneedle device of claim 2, wherein said microneedle is connected to a fluid reservoir storing fluids to be delivered across the biological barrier.

26. The microneedle device of claim 25, wherein said fluid reservoir generates a positive pressure to force the fluids into the microneedle.

27. The microneedle device of claim 26, wherein said positive pressure is generated after the penetration of said microneedle tip into the biological barrier.

28. The microneedle device of claim 2, wherein said microneedle is connected to a fluid reservoir for storing fluids extracted below the surface of the biological barrier.

29. The microneedle device of claim 28, wherein said fluid reservoir generates a negative pressure to extract fluids through the microneedle and from below the penetrated biological barrier.

30. The microneedle device of claim 29, wherein said negative pressure is generated after the penetration of said microneedle tip into the biological barrier.

31. The microneedle device of claim 2, wherein the microneedle tip is tapered.

32. The microneedle device of claim 2, wherein the microneedle tip is blunt.

33. The microneedle device of claim 2, wherein the microneedle tip is serrated.

34. The microneedle device of claim 2, wherein a spiral pattern is disposed on the outer surface of the microneedle tip.

35. The microneedle device of claim 2, wherein the microneedle tip is made of glass and covered with a plastic material.

36. The microneedle device of claim 2, wherein the microneedle tip is transparent/translucent.

37. The microneedle device of claim 2, further including a suction cup or mechanical stretching device to stretch the biological barrier to facilitate penetration by the microneedle tip.

38. The microneedle device of claim 2, wherein said insert comprises a plurality of through-bores, each configured to receive one additional microneedle, said microneedles are so arranged for rotating about a common longitudinal axis.

39. The microneedle device of claim 38, wherein the tips of said microneedles are so arranged to converge to the same area.

40. The microneedle device of claim 38, wherein each of said microneedles is independently connected to its own fluid reservoir.

41. The microneedle device of claim 40, wherein at least two of said fluid reservoirs contain different fluids.

42. The microneedle device of claim 2, wherein said insert comprises a plurality of through-bores, each configured to receive one additional microneedle, said microneedles are so arranged for rotating about their own longitudinal axis.

43. The microneedle device of claim 42, further comprising a drive to commonly drive at least two of said microneedles.

44. The microneedle device of claim 43, wherein the drive includes a common drive shaft with a gear wheel that engages with gear wheels disposed on the commonly driven microneedles.

45. The microneedle device of claim 2, wherein the microneedle is made of glass, silicon, or metal.

46. The microneedle device of claim 2, wherein the microneedle is made of a transparent or translucent material.

47. The microneedle device of claim 2, wherein the microneedle is coated with a plastic or polymer layer.

48. The microneedle device of claim 2, wherein the maximum penetration depth into the biological barrier is less than 1 mm or 500 μm.
